(19) United States
(12) Patent Application Publication
(10) Pub. No.: US 2026/0151374 A1
(43) Pub. Date: Jun. 4, 2026

(54) Cinnamobactin and Use Thereof in Treatment of Bacterial Infection

(71) Applicant: XI'AN SHISONG TECHNOLOGY CO., LTD., Xi'an (CN)

(72) Inventor: Bing Liu, Xi'an (CN)

(21) Appl. No.: 19/408,096

(22) Filed: Dec. 3, 2025

Related U.S. Application Data
(63) Continuation-in-part of application No. PCT/CN2023/109316, filed on Jul. 26, 2023.

(30) Foreign Application Priority Data
Jun. 4, 2023 (CN) 202310650575.7

(51) Int. Cl. A61K 31/4045; A61K 31/185; A61K 31/43; A61K 35/76; A61P 31/04; C07C 259/06; C07D 209/16

(52) U.S. Cl. CPC A61K 31/4045; A61K 31/185; A61K 31/43; A61K 35/76; A61P 31/04; C07C 259/06; C07D 209/16

(57) ABSTRACT

The present disclosure relates to cinnamobactin and the use thereof in the treatment of bacterial infections. The cinnamobactin has the following chemical structural formula (I-1). The cinnamobactin uses cinnamoylhydroxamic acid as a parent nucleus, has a modification group at the para-position or the meta-position of a benzene ring, and can be used for treating bacterial infections.

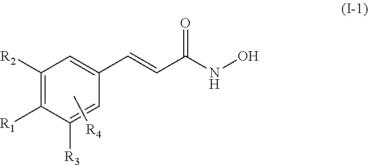

### CROSS-REFERENCE TO RELATED APPLICATIONS

The application is a continuation-in-part application of International Application No. PCT/CN2023/109316, filed on Jul. 26, 2023, which is based on and claims priority to Chinese Patent Application No. 202310650575.7, filed on Jun. 4, 2023. The entire content of disclosures of the above-identified applications are incorporated herein by reference.

### FIELD

The present disclosure relates to the technical field of biological medicine, and specifically to a cinnamobactin and use of the same in the treatment of bacterial infections, especially to the cinnamobactin or pharmaceutically acceptable salt thereof for use in treating bacterial infections.

### BACKGROUND

Antimicrobial resistance (AMR) is a major threat to human health. In 2019, approximately 4.95 million deaths worldwide were associated with AMR. In South Asia and sub-Saharan Africa, the number of deaths caused by AMR has exceeded that of any other disease. It is predicted that by 2050, 10 million people will die from AMR globally each year, at which point AMR will surpass cancer to become the leading cause of death in humans.

The AMR crisis is attributed to the overuse of antibiotics and the failure in the development of new drugs. Since the late 1980s, no new classes of antibiotics have been commercialized; therefore, the period from 1987 to the present is also known as the “antibiotic discovery void era”.

Currently, most antibiotics exert their antibacterial effects by disrupting bacterial nucleic acids, cell walls, or protein synthesis. Bacteria have evolved a variety of counterstrategies against these three antibacterial mechanisms. Thus, exploring new antibacterial targets different from the above three and developing new classes of antibacterial drugs are important approaches to addressing bacterial resistance at present.

### SUMMARY

The present disclosure aims at solving one of the technical problems in the related art at least to some extent. Accordingly, the present disclosure provides a cinnamobactin or pharmaceutically acceptable salt thereof for use in treating bacterial infections.

The bacterial histone HU is a highly conserved DNA-binding protein in bacteria. HU is indispensable in bacteria such as Staphylococcus aureus and Mycobacterium tuberculosis, as well as in mycoplasmas and chlamydiae, and its coding gene (hup) cannot be knocked out. In other bacteria, HU acts as a key protein, knocking out the coding gene of which leads to severe bacterial growth defects. HU exists in all stages of bacterial growth and, through binding to DNA, HU maintains the stability of the bacterial nucleoid region, regulates the bacterial expression under stress, and participates in the formation of bacterial biofilms. Some studies have confirmed the feasibility of HU as an antibiotic target. In previous research, the inventors found that the protein Gp46 encoded by bacteriophage SPO1 can bind to HU from different bacteria and inhibit its DNA-binding ability; overexpression of Gp46 in bacteria causes the bacteria to elongate, with disappearance of the nucleoid region, which is similar to phenotypes of bacteria with hup knocked out.

Starting from the molecular mechanism by which the phage protein Gp46 inhibits the bacterial histone HU, the inventors discovered and defined a completely novel class of antibiotics—cinnamobactins, structures of which include two types: i) a class of cinnamohydroxamic acid derivatives with cinnamohydroxamic acid as a parent nucleus and R groups at para or meta positions of the benzene ring; and ii) a class of cinnamaldehyde derivatives with cinnamaldehyde as a parent nucleus and R groups at para or meta positions of the benzene ring. Cinnamobactins have the following two types of chemical structures:

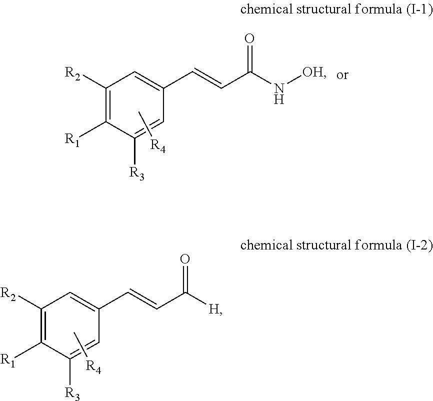

where the chemical structural formula (I-1) represents the cinnamohydroxamic acid derivatives, while the chemical structural formula (I-2) represents the cinnamaldehyde derivatives. The inventors has made an innovative discovery that cinnamobactins can be used to treat bacterial infections, thereby serving as a drug for treating diseases caused by bacterial infections.

Specifically, the present disclosure provides the following solutions.

In a first aspect, there is provided in embodiments of the present disclosure use of a cinnamobactin with the following structural formula or a pharmaceutically acceptable salt thereof in the preparation of a medicament for treating a bacterial infection,

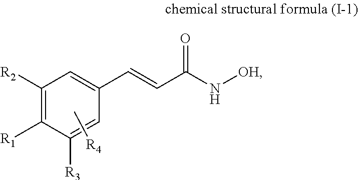

where R₁, R₂ and R₃ each are hydrogen or a negatively charged substituent group, where if R₁ is hydrogen, R₂ and R₃ each are hydrogen or a negatively charged substituent group, and if R₁ is a negatively charged substituent group, R₂ and R₃ each are hydrogen; and
R₄ is hydrogen, fluorine, chlorine, bromine, iodine, methyl, methoxy, hydroxyl, nitryl, amino or substituted amino.

According to embodiments of the present disclosure, said R₁, R₂ and R₃ are each independently selected from hydrogen, fluorine, chlorine, bromine, nitryl, methoxy, aldehyde, carboxyl,

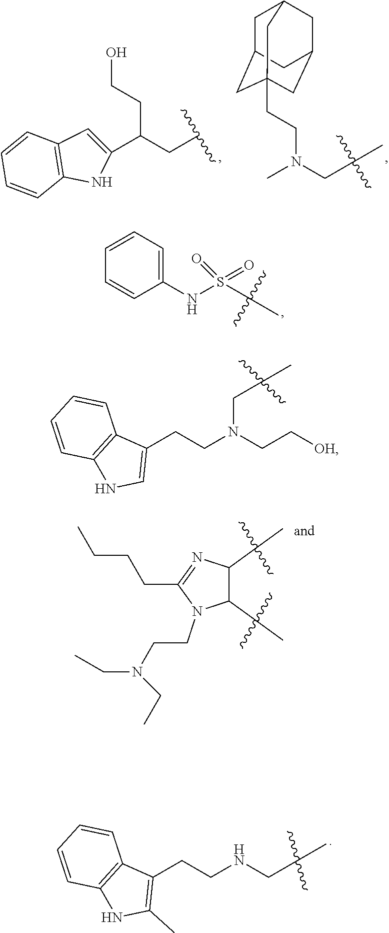 [SMILES: *CC(CCO)c1cc2ccccc2[nH]1 ; *CN(C)CCC12CC3CC(CC(C3)C1)C2 ; *S(=O)(=O)Nc1ccccc1 ; *CN(CCO)CCc1c[nH]c2ccccc12 ; *C1N=C(CCCC)N(CCN(CC)CC)C1* ; *CNCCc1c(C)[nH]c2ccccc12]

According to embodiments of the present disclosure, the cinnamobactin is at least one of the following compounds:

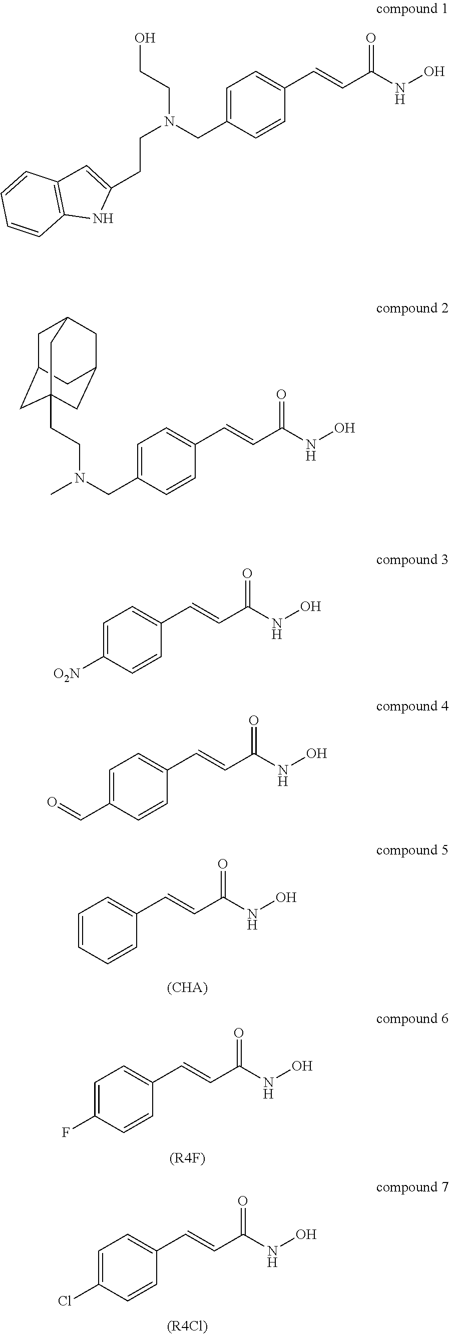

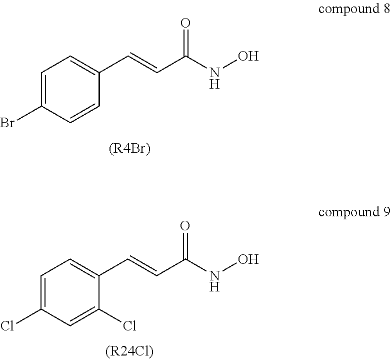

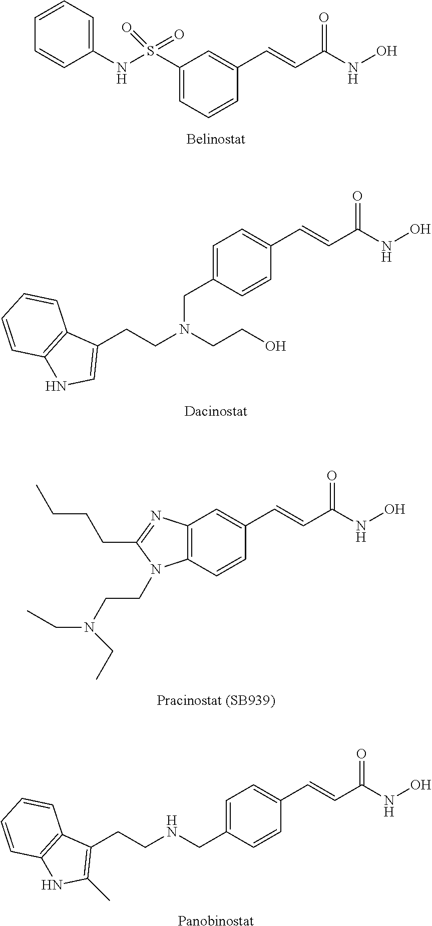

According to embodiments of the present disclosure, the bacteria are at least one of Staphylococcus aureus, methicillin-resistant and multidrug-resistant Staphylococcus aureus, Mycobacterium tuberculosis, multidrug-resistant Mycobacterium tuberculosis, Bacillus subtilis, Escherichia coli, Klebsiella pneumoniae, Acinetobacter baumannii, and Pseudomonas aeruginosa.

According to embodiments of the present disclosure, the cinnamobactin is with a minimal inhibitory concentration of less than 250 μg/ml against the bacteria.

In a second aspect, there is provided in embodiments of the present disclosure a compound with the following chemical structural formula or a pharmaceutically acceptable salt thereof,

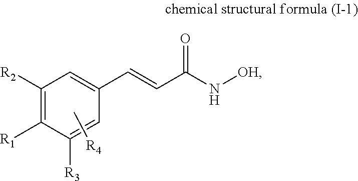

where R₁ is hydrogen, fluorine, chlorine, bromine, nitryl, methoxy, aldehyde, carboxyl,

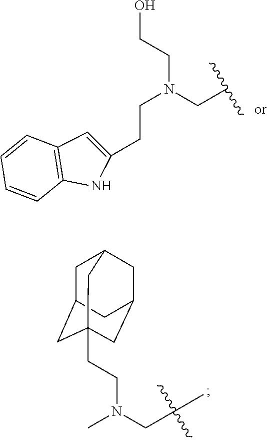 [SMILES: *CN(CCO)CCc1cc2ccccc2[nH]1 ; *CN(C)CCC12CC3CC(CC(C3)C1)C2]

R₂ and R₃ are hydrogen; and
R₄ is selected from hydrogen, fluorine, chlorine, bromine or iodine.

According to embodiments of the present disclosure, the compound is at least one of the following compounds:

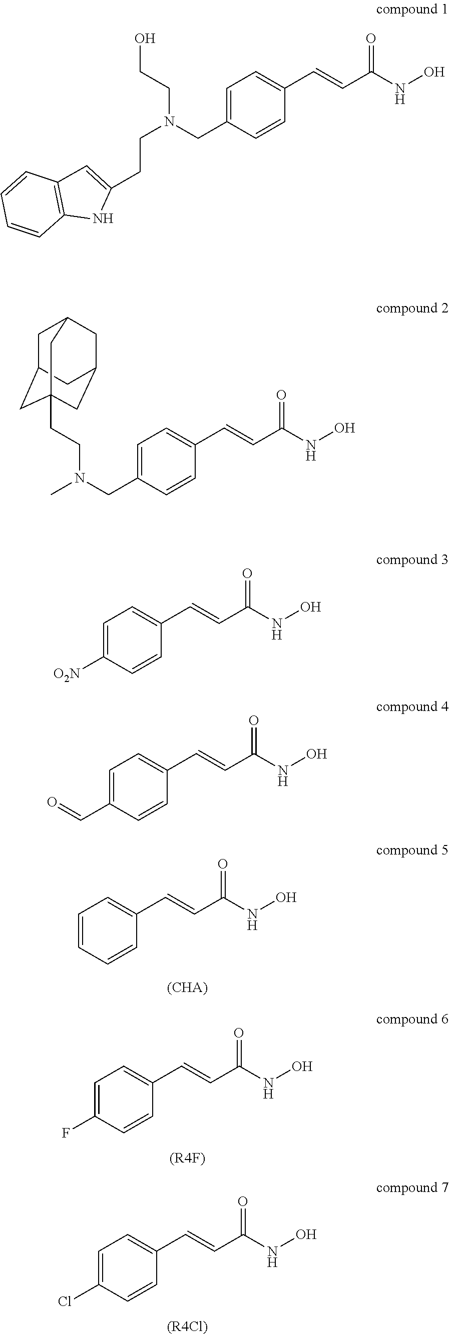

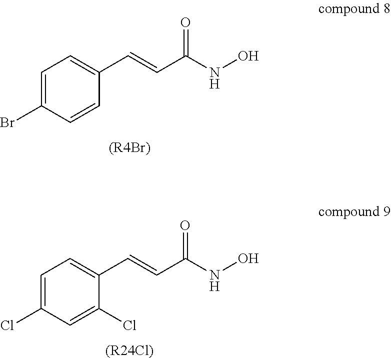

In a third aspect, there is provided in embodiments of the present disclosure a pharmaceutical composition, including a compound or a pharmaceutically acceptable salt thereof according to any one of embodiments in the second aspect of the present disclosure and a pharmaceutically acceptable carrier.

In a fourth aspect, there is provided in embodiments of the present disclosure use of a compound or a pharmaceutically acceptable salt thereof according to any one of embodiments in the third aspect of the present disclosure in the preparation of a medicament for treating a bacterial infection.

According to embodiments of the present disclosure, the compound or pharmaceutically acceptable salt thereof exerts bacteriostasis through binding to a Pit domain of a bacterial histone HU.

In a fifth aspect, there is provided in embodiments of the present disclosure a method for treating a disease caused by a bacterial infection, including: administering a therapeutically effective amount of a cinnamobactin with the following structural formula or a pharmaceutically acceptable salt thereof to a subject in need,

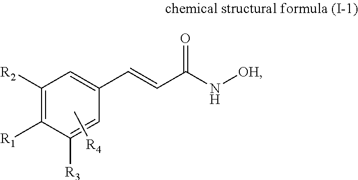

where R₁, R₂ and R₃ each are hydrogen or a negatively charged substituent group, where if R₁ is hydrogen, R₂ and R₃ each are hydrogen or a negatively charged substituent group, and if R₁ is a negatively charged substituent group, R₂ and R₃ each are hydrogen; and
R₄ is hydrogen, fluorine, chlorine, bromine, iodine, methyl, methoxy, hydroxyl, nitryl, amino or substituted amino.

The cinnamobactin exerts bacteriostasis through binding to a Pit domain of a bacterial histone HU, thereby serving as a drug for treating diseases caused by bacterial infections.

In a sixth aspect, there is provided in embodiments of the present disclosure a method for binding to a Pit domain of a bacterial histone HU to treat a bacterial infection or a disease caused by the bacterial infection, including administering to a subject in need a therapeutically effective amount of a cinnamobactin or a pharmaceutically acceptable salt thereof as presented in any one embodiments of the present disclosure.

According to embodiments of the present disclosure, the method includes administering to the subject in need a therapeutically effective amount of the cinnamobactin or a pharmaceutically acceptable salt thereof by oral administration, subcutaneous injection, peritoneal administration, intravenous injection, intramuscular injection, intradural injection, sublingual administration, buccal administration, intrarectal insertion, intravaginal injection, ocular administration, ear administration, nasal administration, inhalation, spraying via the mouth or nose, skin administration or transdermal administration.

According to embodiments of the present disclosure, the cinnamobactin or a pharmaceutically acceptable salt thereof is administered 1 to 5 times a day, such as 1 to 3 times a day, 1 to 2 times a day, or once a day.

According to embodiments of the present disclosure, the subject is a mammal.

According to embodiments of the present disclosure, the method further includes administering an additional therapeutic agent to the subject, simultaneously or sequentially with the cinnamobactin or a pharmaceutically acceptable salt thereof, where the additional therapeutic agent is effective against the bacteria.

According to embodiments of the present disclosure, the additional therapeutic agent includes an antibiotic or a bacteriophage.

According to embodiments of the present disclosure, the antibiotic comprises ampicillin or rifampicin.

In a seventh aspect, there is provided in embodiments of the present disclosure a method for preventing bacterial resistance against a therapeutic agent, including: administering the therapeutic agent to a subject, simultaneously or sequentially with the cinnamobactin or a pharmaceutically acceptable salt thereof.

According to embodiments of the present disclosure, the therapeutic agent includes an antibiotic, such as ampicillin or rifampicin, or a bacteriophage.

The present disclosure obtains beneficial effects as follows.

The present disclosure has made an innovative discovery that a class of compounds, cinnamobactins, can be newly used to treat bacterial infections, thereby serving as a drug for treating diseases caused by bacterial infections. Moreover, these cinnamobactins have strong bacteriostasis, with a minimal inhibitory concentration of less than 250 g/ml, less than 200 μg/ml, less than 150 μg/ml, less than 100 μg/ml, less than 50 μg/ml, less than 20 μg/ml, or even less than 10 μg/ml against the bacteria. Further, the cinnamobactins may be administered in combination with other therapeutic agent such as an antibiotic or a bacteriophage, which can enhance the killing effect on bacteria and prevent the development of bacterial drug resistance.

### BRIEF DESCRIPTION OF THE DRAWINGS

FIG. 1 shows simulation results according to Examples of the present disclosure, where Panel A is a schematic diagram showing a binding between the protein Gp46 from bacteriophage and the bacterial histone HU; Panel B is a schematic diagram showing the virtual screening results of the two targets.

FIG. 2 shows respective antibiotic susceptibility test results of belinostat, panobinostat, pracinostat, dacinostat, R4Cl, R4Br, R24Cl, R4F, and CHA against a standard strain of Staphylococcus aureus ATCC 29213 according to Examples of the present disclosure.

FIG. 3 shows respective MICs of panobinostat, R4Cl, R24Cl, CHA, R4F and R4Br against the standard strain of Staphylococcus aureus ATCC 29213 according to Examples of the present disclosure.

FIG. 4 shows the morphology and nucleoid region of the standard strain of Bacillus subtilis ATCC 6051 and the standard strain of Staphylococcus aureus ATCC 20213 with and without R4Cl treatment according to Examples of the present disclosure.

FIG. 5 shows HSQC spectrum according to Examples of the present disclosure, where Panel A is a schematic diagram showing the backbone assignment of SaHU by NMR; Panel B shows the result of the NMR titration experiment between SaHU and panobinostat; Panel C is for the NMR titration experiment between SaHU and R4Cl.

FIG. 6 shows complex models according to Examples of the present disclosure, where Panel A shows the main SaHU-drug binding region; Panel B shows the complex model of R4Cl bound to SaHU; and Panel C illustrates the molecular mechanism of cinnamobactin-mediated bacteriostasis.

FIG. 7 shows respective MICs of Panobinostat and R4Cl against commonly clinical bacteria according to Examples of the present disclosure, where Panel A presents the MIC results of panobinostat against the 50 clinically isolated MRSA strain; Panel B presents the MIC results of compound 7 (R4Cl) against the same; Panel C shows MICs of Panobinostat and R4Cl against a standard strain of Mycobacterium tuberculosis ATCC H37Rv and multidrug-resistant Mycobacterium tuberculosis XDR-TB; Panel D shows the MIC results of panobinostat against Acinetobacter baumannii ATCC BBA-1605, Escherichia coli MG1655, a standard strain of Klebsiella pneumoniae ATCC 13883 and Pseudomonas aeruginosa PAO1; and Panel E shows the MIC results of R4Cl against the same.

FIG. 8 shows therapeutic effects of cinnamobactins on mouse skin bacterial infections according to Examples of the present disclosure, where Panel A shows the statistical results of the bacterial load in the skin wounds of mice in each group, and Panel B shows the HE staining results of the skin wounds of mice in each group.

FIG. 9 shows the antibacterial activity and toxicity of R4Cl according to Examples of the present disclosure, where panel A shows the HE staining results of the major organs of mice in each group, panel B shows the statistical results of the bacterial load on the skin wounds of mice in each group, Panel C shows the HE staining results of the skin wounds of mice in each group, and panels D and E show the survival rate curves of sepsis model mice after injection of belinostat (D) and R4Cl (E), respectively.

FIG. 10 shows the anti-biofilm property of R4Cl according to Examples of the present disclosure: (A) Log₂ (intensity) values of SaHU in different fractions as determined by mass spectrometry. Data represent mean±SEM, n=3. (B) Inhibition of P. aeruginosa biofilm formation when R4Cl was added at the time of bacterial inoculation. Top: Crystal violet assay with a representative image shown above the graph. Negative control (Control): 1% DMSO. Statistical significance was determined by one-way ANOVA. Data are presented as mean±SEM from three technical replicates (n=3). ****p<0.0001, ***p<0.001, **p<0.01, ns=not significant vs. control. Bottom: SEM images of control and R4Cl-treated biofilms. Scale bar: 20 μm. (C) Eradication of preformed biofilms of S. aureus and P. aeruginosa by R4Cl added after biofilm maturation. Top: Crystal violet assays with representative images shown above the graphs. Negative control (Control): 1% DMSO. Statistical significance was determined by one-way ANOVA. Data represent mean±SEM from three technical replicates (n=3). ****p<0.0001, ***p<0.001, **p<0.01, ns=not significant vs. control. Bottom: SEM images of control and R4Cl-treated biofilms. Scale bar: 20 μm. (D) Changes in B-DNA and Z-DNA abundance in the presence of 20 μg/mL (101 μM) R4Cl. Fluorescence intensity (FI) was quantified using ImageJ as the ratio to untreated biofilms and normalized to total biomass (FM4-64 signal). Statistical significance was determined by unpaired t-tests. Data represent mean±SEM, n=7. ****p<0.0001, ***p<0.001, *p<0.05.

FIG. 11 shows the DNA superhelical structures of E. coli (A) and Bacillus subtilis (B) after R4Cl treatment, according to Examples of the present disclosure.

FIG. 12 shows the synergistic enhancement of R4Cl in combination with other antibiotics, where panel A shows the morphology of MG1655 after treatment with R4Cl, ampicillin (CB), or their combination; and panels B-C show the transcriptome analysis results of MG1655 after treatment with R4Cl, ampicillin (CB), or their combination; and panel D shows Mycobacterium tuberculosis inhibiting by R4Cl in combination with rifampicin.

FIG. 13 shows the synergistic enhancement of R4Cl in combination with bacteriophage on resistance developing of bacteria.

### DETAILED DESCRIPTION

Reference will now be made in detail to embodiments, examples of which are illustrated in the accompanying drawings. The embodiments described herein with reference to drawings are explanatory, illustrative, and used to generally understand the present disclosure. The embodiments shall not be construed to limit the present disclosure.

Based on the molecular mechanism of bacteriophage-mediated bacteriostasis in nature, this disclosure employs computer-aided drug screening, and utilizes biochemical, cell biological, and animal experiments to discover and verify the bacteriostatic effect of cinnamohydroxamic acid derivatives or cinnamaldehyde derivatives modified at the para position (R4 position) or meta position (R3 position) of the benzene ring. This class of compounds is named Cinnamobactins. Among them, the cinnamohydroxamic acid derivatives modified at the para position of the benzene ring exhibit the best bacteriostatic effect. The provided Cinnamobactins can be used in the preparation of drugs for treating diseases caused by bacterial infections.

The cinnamobactins as described in the present disclosure have structures with two types: i) a class of cinnamohydroxamic acid derivatives with cinnamohydroxamic acid as a parent nucleus and R groups at para position (i.e., R4) or meta position (i.e., R3) of the benzene ring, especially ones with R groups at para position of the benzene ring; and ii) a class of cinnamaldehyde derivatives with cinnamaldehyde as a parent nucleus and R groups at para position (i.e., R4) or meta position (i.e., R3) of the benzene ring, especially ones with R groups at para position of the benzene ring. It should be noted that the term “R4” or “R3” merely refers to a position of the benzene ring, regardless of the substituent group.

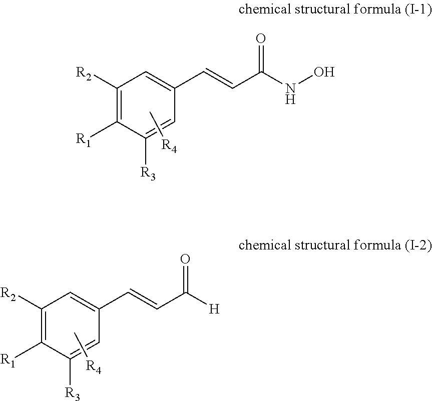

The chemical structural formula (I-1) represents the cinnamohydroxamic acid derivatives, while the chemical structural formula (I-2) represents the cinnamaldehyde derivatives.

The cinnamobactins as described in the present disclosure exert bacteriostasis through binding to bacteria at a site 1, a Pit domain of the bacterial histone HU. The term “negatively charged substituent group” as described in the present disclosure refers to a group with a certain negative charge.

The present disclosure provides in embodiments use of a cinnamobactin with the following structural formula or a pharmaceutically acceptable salt thereof in the preparation of a medicament for treating a bacterial infection,

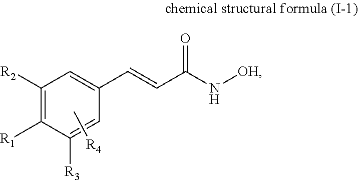

where R₁, R₂ and R₃ each are hydrogen or a negatively charged substituent group, where if R₁ is hydrogen, R₂ and R₃ each are hydrogen or a negatively charged substituent group, and if R₁ is a negatively charged substituent group, R₂ and R₃ each are hydrogen; and
R₄ is hydrogen, fluorine, chlorine, bromine, iodine, methyl, methoxy, hydroxyl, nitryl, amino or substituted amino.

According to specific embodiments of the present disclosure, said R₁, R₂ and R₃ are each independently selected from hydrogen, fluorine, chlorine, bromine, nitryl, methoxy, aldehyde, carboxyl,

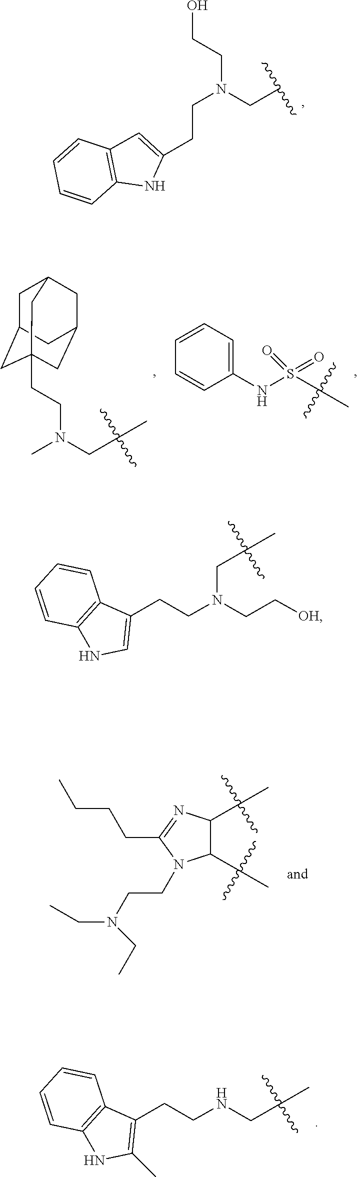 [SMILES: *CN(CCO)CCc1cc2ccccc2[nH]1 ; *CN(C)CCC12CC3CC(CC(C3)C1)C2 ; *S(=O)(=O)Nc1ccccc1 ; *CN(CCO)CCc1c[nH]c2ccccc12 ; *C1N=C(CCCC)N(CCN(CC)CC)C1* ; *CNCCc1c(C)[nH]c2ccccc12]

According to specific embodiments of the present disclosure, the cinnamobactin is at least one of the following compounds:

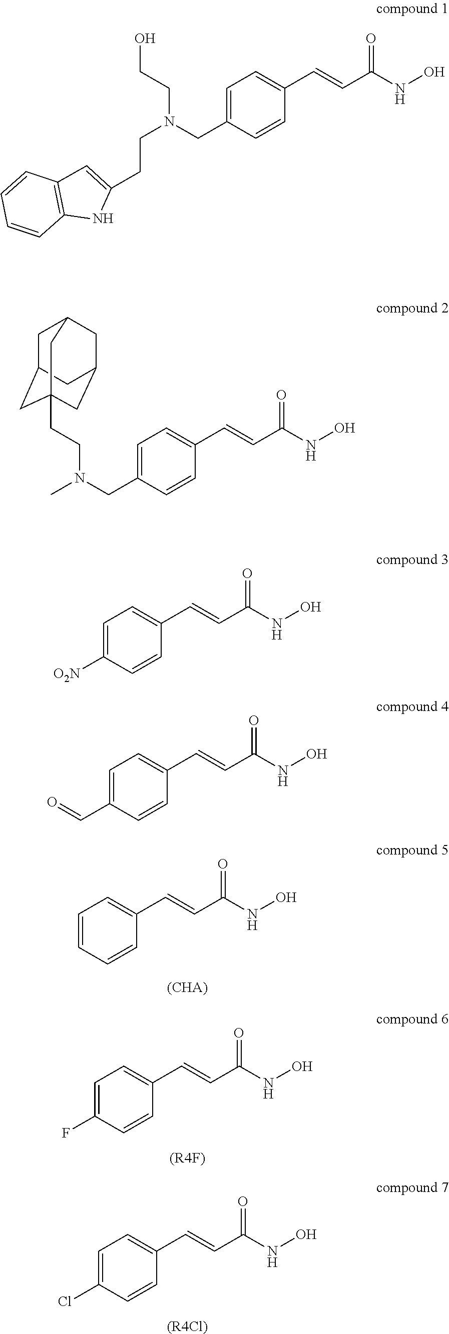

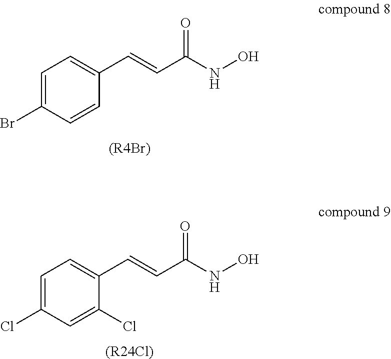

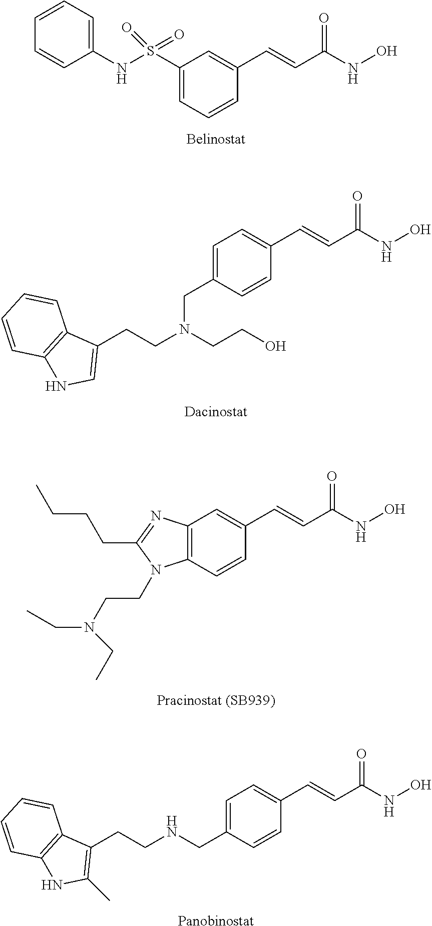

The cinnamobactins provided herein present antibacterial effects on a variety of common clinical bacterial species, including but not limited to Staphylococcus aureus, methicillin-resistant and multidrug-resistant Staphylococcus aureus, Mycobacterium tuberculosis, multidrug-resistant Mycobacterium tuberculosis, Acinetobacter baumannii, Escherichia coli, Klebsiella pneumoniae, Pseudomonas aeruginosa, and etc. The cinnamobactin provided herein is of a minimal inhibitory concentration, against pathogenic bacteria, of less than 300 μg/ml, less than 250 μg/ml, less than 220 μg/ml, less than 200 μg/ml, less than 180 μg/ml, less than 150 μg/ml, less than 130 μg/ml, less than 100 μg/ml, less than 90 μg/ml, less than 80 μg/ml, less than 70 μg/ml, less than 60 μg/ml, less than 50 μg/ml, less than 40 μg/ml, less than 30 μg/ml, less than 20 μg/ml, less than 10 μg/ml, less than 9 μg/ml, less than 8 μg/ml, less than 7 μg/ml, less than 6 μg/ml, less than 5 μg/ml, less than 4 μg/ml, less than 3 μg/ml, less than 2 μg/ml, less than 1 μg/ml, etc.

The present disclosure further provides in embodiments a compound with the following chemical structural formula or a pharmaceutically acceptable salt thereof,

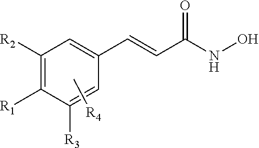

chemical structural formula (I-1),
where R₁ is hydrogen, fluorine, chlorine, bromine, nitryl, methoxy, aldehyde, carboxyl,

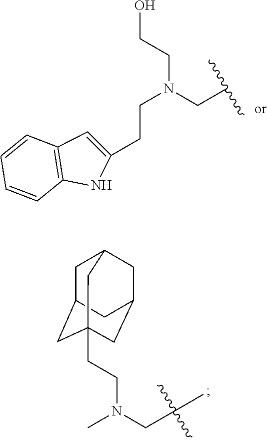 [SMILES: *CN(CCO)CCc1cc2ccccc2[nH]1 ; *CN(C)CCC12CC3CC(CC(C3)C1)C2]

R₂ and R₃ are hydrogen; and
R₄ is selected from hydrogen, fluorine, chlorine, bromine or iodine.

According to embodiments of the present disclosure, the compound is at least one of the following compounds:

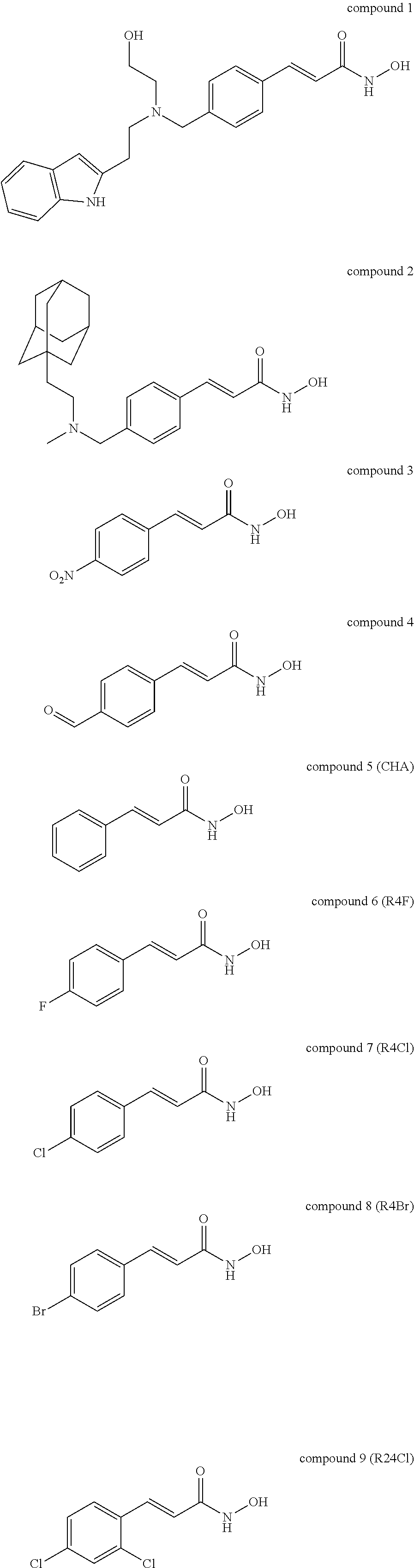

The present disclosure further provides in embodiments a pharmaceutical composition, including a compound or a pharmaceutically acceptable salt thereof as described above and a pharmaceutically acceptable carrier.

The term “pharmaceutically acceptable” refers to compounds, materials, compositions, and/or dosage forms that, within the scope of sound medical judgment, are suitable for use in contact with the tissues of humans and animals, without excessive toxicity, irritation, allergic response, or other problems or complications, and that are commensurate with a reasonable benefit/risk ratio.

The term “pharmaceutically acceptable salts” refers to salts of the compounds disclosed herein, which are prepared from the compounds with specific substituents discovered in the present disclosure or from relatively non-toxic acids or bases. In the case of that the compounds of the present disclosure contain relatively acidic functional groups, base addition salts may be obtained by contacting the neutral form of such compounds with a sufficient amount of a base in a pure solution or a suitable inert solution. Examples of pharmaceutically acceptable salts include inorganic acid salts, organic acid salts, and the like.

The compound or pharmaceutical composition of the present disclosure may be administered to a subject in need via various routes. All methods of administration may be contemplated, but the pharmaceutical composition may be administered by, for example, oral administration, subcutaneous injection, peritoneal administration, intravenous injection, intramuscular injection, paraspinal space (intradural) injection, sublingual administration, buccal administration, intrarectal insertion, intravaginal injection, ocular administration, ear administration, nasal administration, inhalation, spraying via the mouth or nose, skin administration, transdermal administration, and the like.

Further, the compound or pharmaceutical composition according to the present disclosure may be administered to a subject in need thereof 1 to 10 times, 1 to 5 times, 1 to 3 times, or 1 to 2 times per day, and may be administered at intervals of 1 to 10 days, 1 to 5 days, 1 to 3 days, or every day, but is not limited thereto.

In some embodiments, the cinnamobactin or a pharmaceutically acceptable salt thereof may be administered as an individual therapeutic agent or may be administered, simultaneously or sequentially, in combination with an additional therapeutic agent being effective against the bacteria, and may be administered in a single dose or multiple doses. In some embodiments, the additional therapeutic agent may be an antibiotic such as ampicillin or rifampicin, or a bacteriophage.

The present disclosure further provides in embodiments use of a compound or a pharmaceutically acceptable salt thereof as described above in the preparation of a medicament for treating a bacterial infection. The inventors has made an innovative discovery that such a compound or a pharmaceutically acceptable salt thereof exerts bacteriostasis through binding to a Pit domain of a bacterial histone HU.

The present disclosure further provides in embodiments a method for treating or preventing a disease caused by a bacterial infection, including: administering a therapeutically effective amount of a cinnamobactin with the following structural formula or a pharmaceutically acceptable salt thereof to a subject in need,

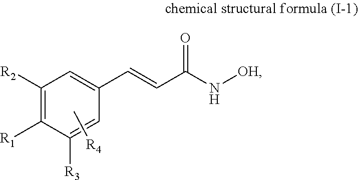

where R₁, R₂ and R₃ each are hydrogen or a negatively charged substituent group, where if R₁ is hydrogen, R₂ and R₃ each are hydrogen or a negatively charged substituent group, and if R₁ is a negatively charged substituent group, R₂ and R₃ each are hydrogen; and
R₄ is hydrogen, fluorine, chlorine, bromine, iodine, methyl, methoxy, hydroxyl, nitryl, amino or substituted amino.

The present disclosure further provides in embodiments a method for binding to a Pit domain of a bacterial histone HU to treat a bacterial infection or a disease caused by the bacterial infection, including administering to a subject in need a therapeutically effective amount of a cinnamobactin with the following structural formula or a pharmaceutically acceptable salt thereof as described in any one embodiments of the present disclosure.

The cinnamobactins by binding to the Pit domain of the bacterial histone HU exerts bacteriostasis, thereby serving as a drug for treating diseases caused by bacterial infections. The mentioned “therapeutically effective amount” can reduce the severity of disease symptoms, increase the frequency and duration of asymptomatic periods of the disease, or alleviate suffering caused by the disease. A “prophylactically effective amount” is generally lower than a therapeutically effective amount. Compared with subjects not treated with the provided compound, the inhibition rate of bacteria in or on the skin surface of the treated subjects reaches more than 10%, more than 15%, more than 20%, more than 25%, more than 30%, more than 40%, more than 45%, more than 50%, more than 55%, more than 60%, more than 65%, more than 70%, more than 75%, more than 80%, more than 85%, or even more than 90% or more than 95%. The mentioned subjects may be either animals or humans. For example, they may be mammals, including cattle, sheep, mice, horses, and the like.

In some embodiments, a dosage may be adjusted according to the severity of the bacteria or an infection caused by the same.

In some embodiments, the disease caused by the bacterial infection may be septicemia/sepsis.

Reference will now be made to the present disclosure through examples. The examples described herein are merely used to illustrate the present disclosure, but shall not be construed to limit the present disclosure. All the compounds shown could be prepared by common knowledge in the art. Among them, the existing compounds in the examples could be directly obtained by purchase, and Compounds 1-9 were prepared with reference to the ZINC20 small molecule library.

### Example 1: Determination of Domains to be Screened and Computer-Aided Drug Screening

The regions on the bacterial histone HU that bind to the protein Gp46 from bacteriophage may be mainly divided into two sites: Site 1 and Site 2. Site 1 is located in the Pit domain of HU, while Site 2 is located in the Arm domain. Site 1 and Site 2 were used as candidate drug target regions. The candidate drugs were from the DrugBank and ZINC20 databases. With the computer software AutoDock Vina, the above candidate regions were each subjected to molecular dynamics simulation calculations, the simulation results of which were sorted by Energy Value (as shown in FIG. 1).

In FIG. 1, Panel A is a schematic diagram showing a binding between the protein Gp46 from bacteriophage and the bacterial histone HU; Panel B is a schematic diagram showing the virtual screening results of the two targets.

With screening, it was discovered and defined a completely novel class of antibiotics—cinnamobactins, structures of which include two types: i) a class of cinnamohydroxamic acid derivatives with cinnamohydroxamic acid as a parent nucleus and R groups at para position (R4) of the benzene ring; and ii) a class of cinnamaldehyde derivatives with cinnamaldehyde as a parent nucleus and R groups at para position (R4) of the benzene ring. Cinnamobactins have the following two types of chemical structures:

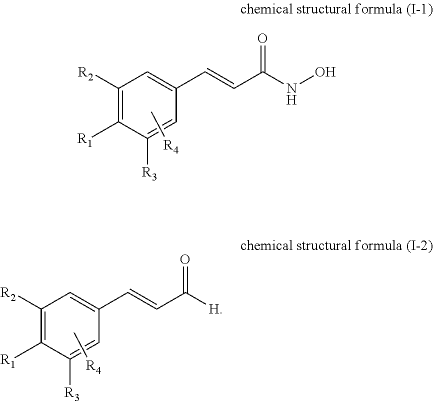

Antibacterial effects of these compounds were investigated by taking Compounds 1-9, as well as Belinostat, Dacinostat, Pracinostat (SB939), and Panobinostat as examples.

### Example 2: Cell-Level Experiments Verify the Inhibitory Effect of Cinnamobactins on Bacteria

At the cellular level, antibacterial properties of the cinnamobactins were preliminarily tested with a disk diffusion method, for investigating respective minimal inhibitory concentrations (MICs) of panobinostat, Compound 5 (CHA), Compound 6 (R4F), Compound 7 (R4Cl), Compound 8 (R4Br), and Compound 9 (R24Cl) against a standard strain of Staphylococcus aureus ATCC 29213. Additionally, the influences of R4Cl on the morphology and nucleoid region of the standard strains of Bacillus subtilis ATCC 6051 and Staphylococcus aureus ATCC 20213 were observed.

The specific experimental procedures and results are as follows.

### 2.1 Antibacterial Test for Cinnamobactins by Disk Diffusion Method

Powders of belinostat, panobinostat, pracinostat, vorinostat, dacinostat, R4Cl, R4Br, R24Cl, R4F, and CHA were each dissolved in DMSO to prepare 10 mg/mL stock solutions. 20 μL of each drug solution or DMSO was added dropwise to blank antibiotic susceptibility disks to prepare drug-containing disks and negative control disks, which were set aside for later use. Among these, vorinostat, a non-cinnamohydroxamic acid derivative, served as a control group for HDAC inhibitors (HDACi).

In accordance with the standards of the Clinical and Laboratory Standards Institute (CLSI), an appropriate amount of colonies was scraped from a fresh LB agar plate culturing the standard strain of Staphylococcus aureus ATCC 29213 for 18-24 hours, and dispersed in LB liquid medium to obtain a bacterial suspension. The turbidity of the suspension was adjusted to 0.5 McFarland units. Within 15 minutes upon preparing the suspension, a sterile swab was dipped into the liquid, then rotated and pressed against the tube wall several times to remove excess bacterial liquid. The swab then streaks a fresh LB agar plate; after each 60° rotation of the plate, streaking was repeated, followed by finally wiping the edge of the plate, a bacterial-seeded plate was obtain. The plate was left to stand for 3-5 minutes to allow complete absorption of the bacterial liquid. After attaching the drug-containing disks to the bacterial-seeded plates, the plates were inverted and incubated in a 37° C. incubator for 16-18 hours before observation. Disks containing DMSO served as negative controls. All tests were performed with 3 biological replicates.

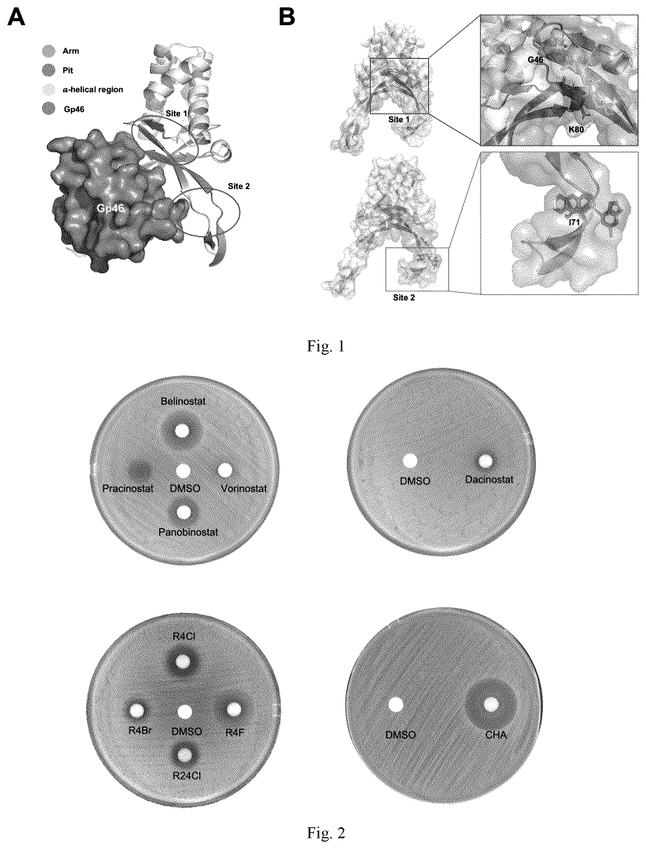

The results are shown in FIG. 2. FIG. 2 presents the antibiotic susceptibility test results of each compound against the standard strain of Staphylococcus aureus ATCC 29213. These experimental results indicated that belinostat, panobinostat, dacinostat, pracinostat (SB939), R4Cl, R4Br, R24Cl, R4F, and CHA exhibited inhibitory effects on Staphylococcus aureus ATCC 29213; in particular, compounds such as belinostat, panobinostat, R4F, and CHA showed better inhibitory effects. 2.2 Investigation into respective minimal inhibitory concentrations (MICs) of panobinostat, R4Cl, R24Cl, CHA, R4F and R4Br against the standard strain of Staphylococcus aureus ATCC 29213.

An appropriate amount of DMSO was added to respective powders of panobinostat, R4Cl, R24Cl, CHA, R4F, and R4Br to prepare high-concentration drug stock solutions. An appropriate volume of each drug stock solution or DMSO was mixed with LB liquid medium to prepare drug-containing LB liquid medium and DMSO-containing LB liquid medium, which were set aside for later use.

The test was conducted in a 96-well plate. In accordance with CLSI standards, an appropriate amount of colonies was scraped from a fresh LB agar plate culturing the standard strain of Staphylococcus aureus ATCC 29213 for 18-24 hours and dispersed in LB liquid medium to obtain a bacterial suspension. The turbidity of the suspension was adjusted to a bacterial density of 1×10⁶ CFU/mL. The bacterial suspension (100 μL) was mixed with 100 μL of drug-containing LB liquid medium, and the mixture was added to a 96-well plate at 200 μL per well, with 3 replicate wells per group (experimental group). The bacterial suspension (100 μL) was mixed with 100 μL of DMSO-containing LB liquid medium, and the mixture was added to the 96-well plate at 200 μL per well, with 3 replicate wells per group (control group). Additionally, 200 μL of LB liquid medium alone was used as a blank control. After the 96-well plates were statically incubated overnight at 37° C., the OD₆₀₀ value of each well was measured using a microplate reader. All experiments were performed with 3 biological replicates.

The results are shown in FIG. 3. FIG. 3 presents the MICs of compounds including panobinostat, R4Cl, R24Cl, CHA, R4F and R4Br against the standard strain of Staphylococcus aureus ATCC 29213. The results in FIG. 3 indicated that these compounds could inhibit Staphylococcus aureus even at low concentrations; in particular, compounds such as R4Cl, R24Cl, and R4Br exhibited better inhibitory effects on Staphylococcus aureus.

### Example 3: Effects of Cinnamobactins on Bacterial Morphology and Nucleoid Region Using Gram Staining and Transmission Electron Microscopy (TEM)

The effects of cinnamobactins on bacterial morphology and nucleoid region were observed with gram stain and TEM. The specific procedures are as follows.

DMSO (1 mL) was added to 24 mg of R4Cl powder to prepare an R4Cl solution.

One drug-treated group and one blank control group were set up for each bacterial strain. Cultures of a standard strain of Bacillus subtilis (B. subtilis) ATCC 6051 and a standard strain of Staphylococcus aureus (S. aureus) ATCC 20213 which were with shaking culture at 37° C. overnight in an incubator were each inoculated into 30 mL of fresh LB liquid medium at a ratio of 1:100 (V/V), with 2 replicate tubes per strain. The inoculated tubes were placed in a 37° C. incubator for shaking culture until their OD₆₀₀ reached approximately 0.4. For the drug-treated group, 500 μL of the R4Cl solution were added to 30 mL of the bacterial culture; for the blank control group, 500 μL of DMSO were added to 30 mL of the bacterial culture. The cultures were continuously shake-incubated at 37° C. for 1 hour and then removed for subsequent use.

### 3.1 Gram Staining and Observation

Bacterial culture (1 mL) was taken from each of the aforementioned drug-treated groups and blank control groups. Gram staining was performed using a Gram Staining Kit (Manufacturer: Solarbio, Cat #G1060-4) following the standard bacterial staining protocol. Bacterial morphology was observed under a microscope. All experiments were conducted with 3 biological replicates.

### 3.2 TEM Sample Preparation and Observation

Bacteria in each group were centrifuged at 4,000 rpm for 10 minutes. After removing the supernatant, the bacterial pellets were fixed with 1 mL/dL glutaraldehyde for 1 hour at 4° C., rinsed 3 times with PBS, and then subjected to osmium acid fixation, gradient dehydration with a series of acetone-ethanol concentrations, epoxy resin embedding, ultra-thin sectioning, and lead-uranium electron staining. Observations were made using a JEM-1400 FLASH transmission electron microscope.

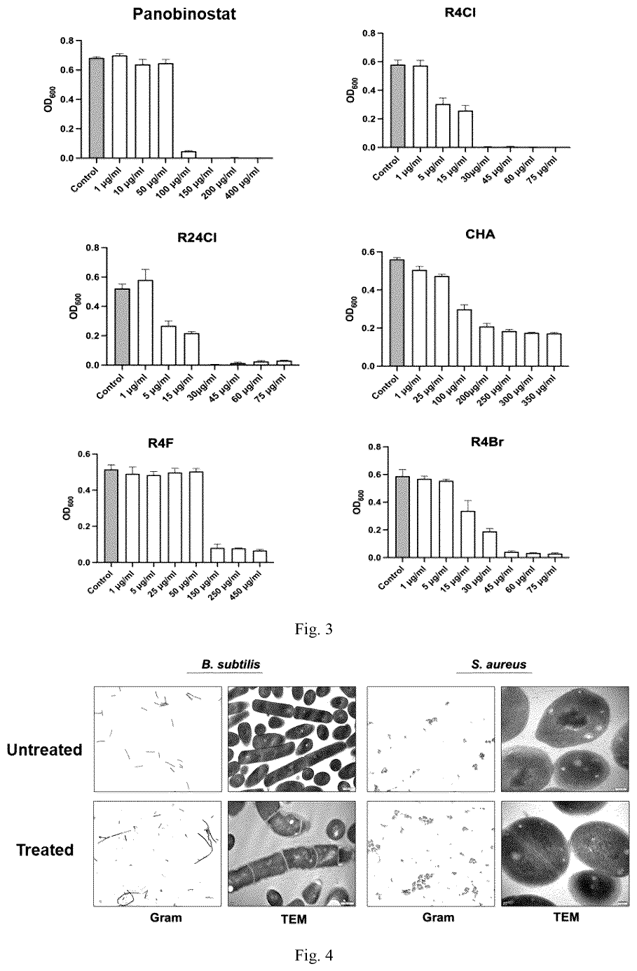

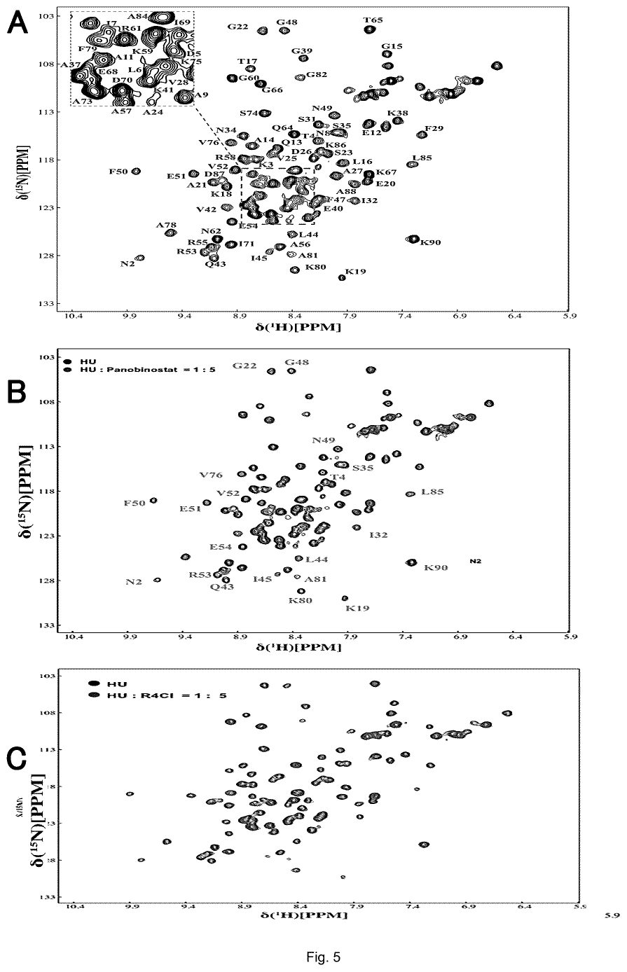

The experimental results are shown in FIG. 4. FIG. 4 presents the morphology and nucleoid region of the standard strain of Bacillus subtilis ATCC 6051 and the standard strain of Staphylococcus aureus ATCC 20213 with and without R4Cl treatment. The results indicated that after R4Cl treatment, the morphology and nucleoid region of Bacillus subtilis and Staphylococcus aureus underwent significant changes compared with the untreated strains. Specifically, the nucleoid region of the treated strains was dispersed, resulting in the bacteria failed to divide, and Bacillus subtilis became elongated.

### Example 4: Construction of the Cinnamobactin-SaHU Protein Complex Model Based on Nuclear Magnetic Resonance (NMR) Experimental Data

At the molecular level, the backbone of SaHU was first assigned via NMR experiments. NMR titration experiments verified the binding of panobinostat or R4Cl with SaHU, and clarified a region SaHU binding to the drug. On this basis, a SaHU-R4Cl complex model was constructed using HADDOCK (v2.2) software.

The specific experimental procedures and results are summarized as follows:

### 4.1 NMR Titration Experiments to Identify the Main SaHU-Cinnamobactin Binding Region

An appropriate amount of DMSO was added to each of panobinostat and R4Cl powders, which were then mixed with a buffer at pH 7.4 containing 50 mM NaCl and 20 mM Tris. This yielded high-concentration drug stock solutions at pH 7.4 containing 50 mM NaCl, 20 mM Tris, and 16% DMSO, which were set aside for later use.

The HSQC spectrum of ¹⁵N-labeled SaHU was recorded on a Bruker 800 MHz NMR spectrometer (Avance III). An appropriate volume of the drug solution was mixed with ¹⁵N-labeled SaHU at a molar ratio of 5:1, to recode the HSQC spectrum again. Changes in the HSQC spectrum before and after drug input were analyzed, and the amino acid residues corresponding to the peaks with significant changes were highlighted on the three-dimensional structure of SaHU, where panobinostat added was marked in purple, and R4Cl added was marked in orange (black-and-white illustrations are provided in FIG. 5).

In FIG. 5, Panel A is a schematic diagram showing the backbone assignment of SaHU by NMR; Panel B shows the result of the NMR titration experiment between SaHU and panobinostat; Panel C is for the NMR titration experiment between SaHU and R4Cl. The experimental results indicated that SaHU bound to both the cinnamobactins, panobinostat and R₄Cl.

### 4.2 Construction of the SaHU-R4Cl Complex Model

Amino acid residues with the most significant changes were identified by calculating the intensity changes of the peaks corresponding to amino acids in the NMR spectrum, and these residues were mapped to the structure of SaHU. Using the surface-exposed residues among them and the small molecule, the SaHU-R4Cl complex model was constructed via HADDOCK.

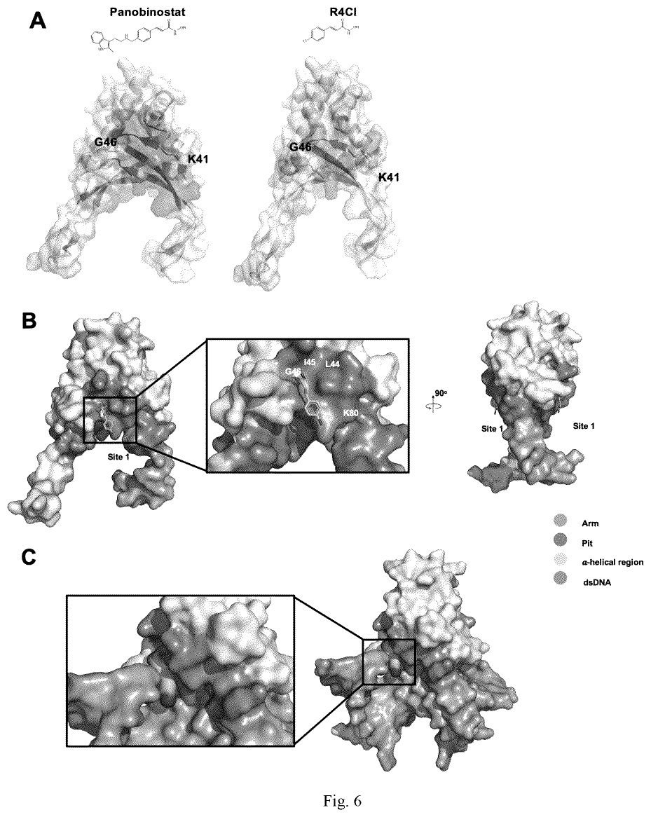

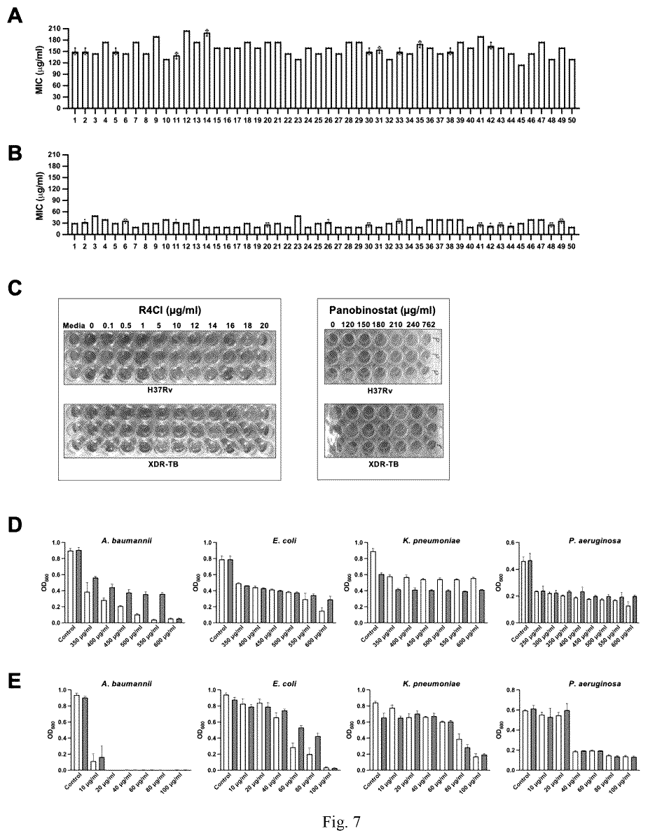

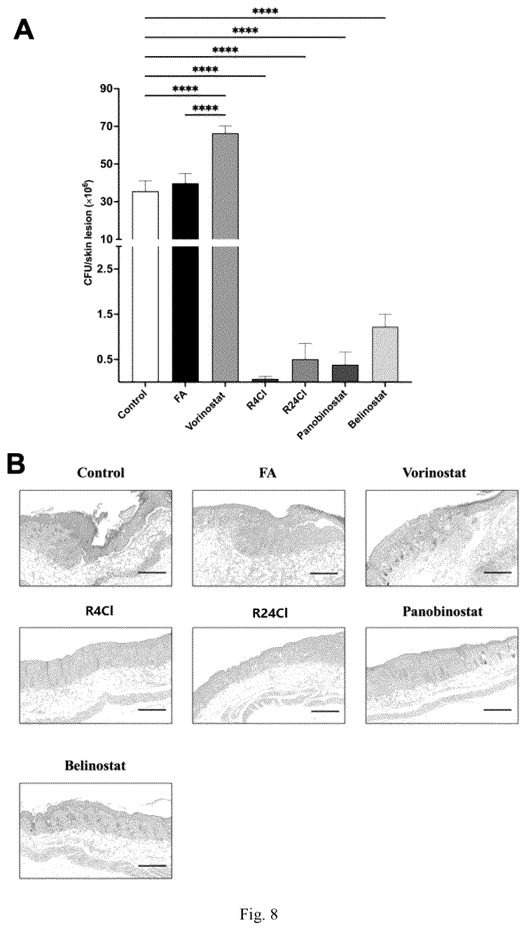

The constructed complex model is shown in FIG. 6. In FIG. 6, Panel A shows the main SaHU-drug binding region; Panel B shows the complex model of R4Cl bound to SaHU; and Panel C illustrates the molecular mechanism of cinnamobactin-mediated bacteriostasis. The results in FIG. 6 reveal that cinnamobactins exert their bacteriostatic effect primarily through Site 1, where the cinnamoylhydroxamic acid group mainly functions to bind to SaHU, while the R₁ group extends from Site 1 and primarily functions to block the binding of SaHU to DNA. Cinnamobactins blocking the binding of HU to DNA directly leads to the phenotypes of nucleoid region dispersion and inability to divide, which is consistent with the previously reported function of SaHU. Through this mechanism, cinnamobactins can exert bacteriostatic effects, thereby enabling the treatment of diseases caused by bacterial infections.

### Example 5: Inhibitory Effect of Cinnamobactins on Clinically Common Bacterial Strains

In Example 5, the minimal inhibitory concentrations (MICs) of panobinostat and R4Cl against clinically common pathogenic bacteria were determined. These pathogenic bacteria include: 50 clinically isolated strains of MRSA (methicillin-resistant and multidrug-resistant Staphylococcus aureus), a standard strain of Mycobacterium tuberculosis ATCC H37Rv, multidrug-resistant Mycobacterium tuberculosis XDR-TB, a standard strain of Acinetobacter baumannii ATCC BBA-1605, Escherichia coli MG1655, a standard strain of Klebsiella pneumoniae ATCC 13883, and Pseudomonas aeruginosa PAO1.

The specific experimental procedures and results are summarized as follows.

### 5.1 MICs of Panobinostat and R4Cl Against 50 Clinically Isolated MRSA Strains

Procedures refer to the MIC determination of Staphylococcus aureus in Example 2.

The experimental results are shown in Panels A and B of FIG. 7, where Panel A presents the MIC results of panobinostat against the 50 clinically isolated MRSA strains, while Panel B presents the MIC results of R4Cl against the same. The results indicated that both panobinostat and R4Cl exhibited inhibitory effects on the 50 clinically isolated MRSA strains: the MIC of panobinostat was approximately 100-200 μg/mL, while R4Cl showed a better effect with n MIC of approximately 20-50 μg/mL.

5.2 MICs of Panobinostat and R4Cl Against a Standard Strain of Mycobacterium tuberculosis ATCC H37Rv and Multidrug-Resistant Mycobacterium tuberculosis XDR-TB

An appropriate amount of DMSO was added to panobinostat and R4Cl powders each to prepare high-concentration drug stock solutions. An appropriate volume of each drug stock solution or DMSO was mixed with MiddleBrook 7H9 medium to prepare drug-containing MiddleBrook 7H9 medium and DMSO-containing LB liquid medium, which were set aside for later use.

An appropriate amount of colonies was scraped from MiddleBrook 7H10 medium and dispersed in MiddleBrook 7H9 liquid medium to obtain a bacterial suspension. The turbidity of the suspension was adjusted to 1 McFarland unit. An appropriate volume of the bacterial suspension was inoculated into MiddleBrook 7H9 medium at a ratio of 1:200 (v/v) and set aside for later use.

The bacterial suspension (100 μL) was mixed with 100 μL of drug-containing MiddleBrook 7H9 medium, and the mixture was added to a 96-well plate at 200 μL per well, with 3 replicate wells per group (experimental group). The bacterial suspension (100 μL) was mixed with 100 μL of DMSO-containing LB liquid medium, and the mixture was added to the 96-well plate at 200 μL per well, with 3 replicate wells per group (control group). Additionally, 200 μL of MiddleBrook 7H9 medium alone was used as a blank control. After the 96-well plate was statically incubated at 37° C. for 14 days, 25 μL of 0.02% resazurin solution were added to each well for continuously incubation at 37° C. for 24 hours. The lowest drug concentration among wells showing a blue color was determined as the MIC. All experiments were performed with 3 biological replicates.

The results are shown in Panel C of FIG. 7. These experimental results indicated that both panobinostat and R4Cl exhibited inhibitory effects on the standard strain of Mycobacterium tuberculosis ATCC H37Rv and multidrug-resistant Mycobacterium tuberculosis XDR-TB: the MIC of panobinostat against Mycobacterium tuberculosis ATCC H37Rv was approximately 180 μg/mL, with approximately 210 μg/mL against XDR-TB; R4Cl against Mycobacterium tuberculosis ATCC H37Rv was approximately 14 μg/mL, with approximately 10 μg/mL against XDR-TB.

5.3 MICs of Panobinostat and R4Cl Against a Standard Strain of Acinetobacter baumannii ATCC BBA-1605, Escherichia coli MG1655, Standard Strain of Klebsiella pneumoniae ATCC 13883 and Pseudomonas aeruginosa PAO1

Procedures refer to the MIC determination of Staphylococcus aureus in Example 2.

The experimental results are shown in Panels D and E of FIG. 7, where Panel D presents the MIC results of panobinostat against these strains, while Panel E presents the MIC results of R4Cl against the same. These results indicated that both panobinostat and R4Cl exhibited inhibitory effects on all of these strains: the MIC of panobinostat was approximately 350 μg/mL, while R4Cl showed a better effect with a MIC of approximately 10-40 μg/mL.

### Example 6: Animal Experiments Verify the Therapeutic Effect of Cinnamobactins on Skin Bacterial Infections

Based on the aforementioned molecular and cellular experiments, animal experiments were further conducted to verify the inhibitory and therapeutic effects of belinostat, panobinostat, R4Cl, and R24Cl on bacterial infections.

Fusidic acid-resistant MRSA was inoculated into 5 mL of LB liquid medium and cultured with shaking in an incubator at 37° C. The culture was removed for subsequent use when the bacterial density reached 1×10⁷ CFU/mL.

The mouse skin barrier was disrupt by a tape stripping (TAP) method, to establish a mouse model of superficial skin injury. Specifically, on the day of modeling, the dorsal skin of 6-8-week-old female Balb/c mice was depilated with depilatory cream to form a bare skin area of approximately 1 cm². On the same day, mice were anesthetized by intraperitoneal injection of isoflurane at a dose of 10 mL/kg. A medical tape was tightly adhered to the depilated skin area of the mouse by the same operator with approximately the same force, then peeled off. The above adhesion-peeling operation was repeated several times to remove the stratum corneum. The operation was stopped when the mouse skin turned red and shiny without irregular bleeding. The aforementioned bacterial solutions (10 μL) each were evenly applied to the damaged skin area of the mice, thus establishing the mouse model of superficial skin injury with infection maintained for 24 hours.

The mice were randomly divided into 7 groups: a blank control group (Control), fusidic acid administration group (FA Administration Group), vorinostat administration group, R4Cl administration group, R24Cl administration group, panobinostat administration group, and belinostat administration group, with 5 mice in each group. All mice in the blank control group were sacrificed on the day before the start of treatment to quantify the bacterial inoculation amount. Mice in the administration groups were dosed twice a day (at 8:00 a.m. and 8:00 p.m. respectively) for 3 consecutive days after infection; the dose for each of the cinnamobactin groups was 50 μL per mouse per dose, while the dose for the fusidic acid administration group was 0.1 g per mouse per dose. The food intake, water intake, and mental state of the mice were observed at least twice a day. After the experiment, the mice were sacrificed, and the wound area of approximately 1 cm² was immediately excised, which was placed into a tissue homogenization tube together with 1 mL of normal saline and magnetic beads for tissue homogenization. The tissue homogenate (100 μL) was serially diluted 10-fold with normal saline; 100 μL of each diluted solution was evenly spread on an LB agar plate. After inverted incubation overnight for 16-24 hours, colony counting was performed, and the counting results were converted to the bacterial content per square centimeter of tissue (CFU/cm²). A small amount of the wound tissue was excised for hematoxylin and eosin (HE) staining.

The experimental results are shown in FIG. 8. Panel A of FIG. 8 shows the statistical results of the bacterial load in the skin wounds of mice in each group, and Panel B shows the HE staining results of the skin wounds of mice in each group. The results indicated that compared with the control group and the vorinostat administration group, the bacterial load in the skin wounds of mice treated with belinostat, panobinostat, R4Cl, and R24Cl was significantly reduced; moreover, after drug treatment, the skin wounds of the mice in each treatment group became smaller and were effectively alleviated.

### Example 7: Cinnamobactin Exhibits Lower Toxicity and Stronger Antibacterial Activity

This Example further tests the toxicity and bactericidal activity of belinostat and R4Cl (an optimized derivative based on belinostat).

### 7.1 Antibacterial Activity Test

### 7.1.1 Antibacterial Activity on Skin Wounds

A mouse model of superficial skin injury (also referred to as a “mouse skin infection model”) was established following the method described in Example 6.

After establishing the mouse model of superficial skin injury, the mice were randomly divided into groups, including a blank control group, a fusidic acid (FA) administration group (positive control group), and an R4Cl administration group. Each group of mice was treated according to the method in Example 6, with continuous administration for 3 days. For the R4Cl administration group, different dosage gradients were set.

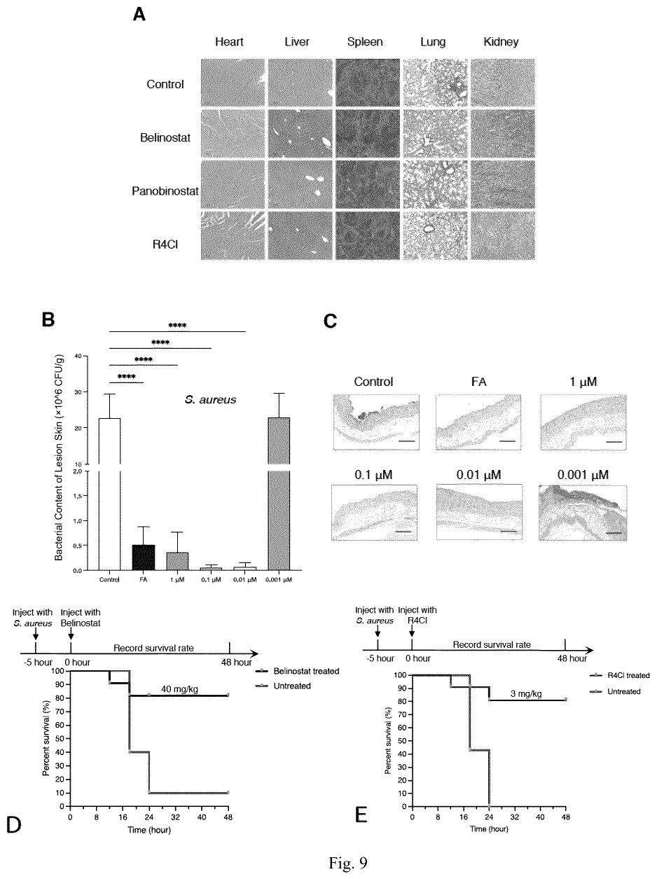

Panel B in FIG. 9 shows the statistical results of the bacterial load on the skin wounds of mice in each group, and Panel C shows the HE staining results of the skin wounds of mice in each group. The results indicated that compared with the blank control group, the bacterial load on the skin wounds of mice treated with R4Cl was significantly reduced. Further, compared with the positive control group, treatment with 1 μM R4Cl showed better efficacy than the FA administration group. When the concentration of R4Cl was diluted to 0.1 μM and 0.01 μM, the bacterial load on the mouse skin wounds decreased significantly (Panel B). Tissue sections revealed that, in the R4Cl treatment groups with different concentration gradients, the epidermis at the skin infection sites of mice was repaired and inflammation was reduced (Panel C).

These results demonstrate that R4Cl has effective bactericidal activity. Moreover, it achieves excellent bactericidal effects at low doses ranging from 0.01 μM to 1 μM. Compared with belinostat and the positive control group, the minimum inhibitory concentration (MIC) of R4Cl is significantly lower.

7.1.2 Antibacterial Activity against Bloodstream Infections

A mouse model of sepsis was established in accordance with the method described in Chen, H. et al., Cinnamic-Hydroxamic-Acid Derivatives Exhibit Antibiotic, Anti-Biofilm, and Supercoiling Relaxation Properties by Targeting Bacterial Nucleoid-Associated Protein HU. Advanced science (Weinheim, Baden-Wurttemberg, Germany), e09876. https://doi.org/10.1002/advs.202509876.

After establishing the mouse sepsis model, the mice were randomly divided into groups, including a blank control group, a belinostat administration group, and an R4Cl administration group. Each group of mice was treated according to the protocol shown in panels D and E of FIG. 9.

Panels D and E of FIG. 9 show the survival rate curves of sepsis model mice after injection of belinostat and R4Cl, respectively. The results indicated that both R4Cl and belinostat could effectively improve the survival rate of septic mice. Notably, compared with belinostat, R4Cl administered at a low dose achieved better efficacy than belinostat administered at a high dose. This suggests that R4Cl is a more potent and safer antibacterial compound.

### 7.2 Toxicity Test

Healthy mice were randomly divided into groups, including a blank control group, a belinostat administration group, a panobinostat administration group, and an R4Cl administration group. Each group of mice received intravenous injections for 7 consecutive days. The blank control group was injected with normal saline, while the other administration groups were injected with the respective drugs at a concentration of 44 mg kg⁻¹ for Belinostat, 28 mg kg⁻¹ for Panobinostat, and 44 mg/kg⁻¹ for R4Cl.

Panel A of FIG. 9 shows the HE staining results of the major organs of mice in each treatment group. The results indicated that after 7 consecutive days of intravenous injection, R4Cl exhibited nonobvious toxic effects on the major organs of mice, similar to belinostat (which has low toxicity). This demonstrates that R4Cl not only has enhanced antibacterial activity but also possesses excellent low-toxicity characteristics.

### Example 8: R4Cl Inhibits the Formation of Bacterial Biofilms and Disrupts Formed Biofilms, to Combat Bacteria.

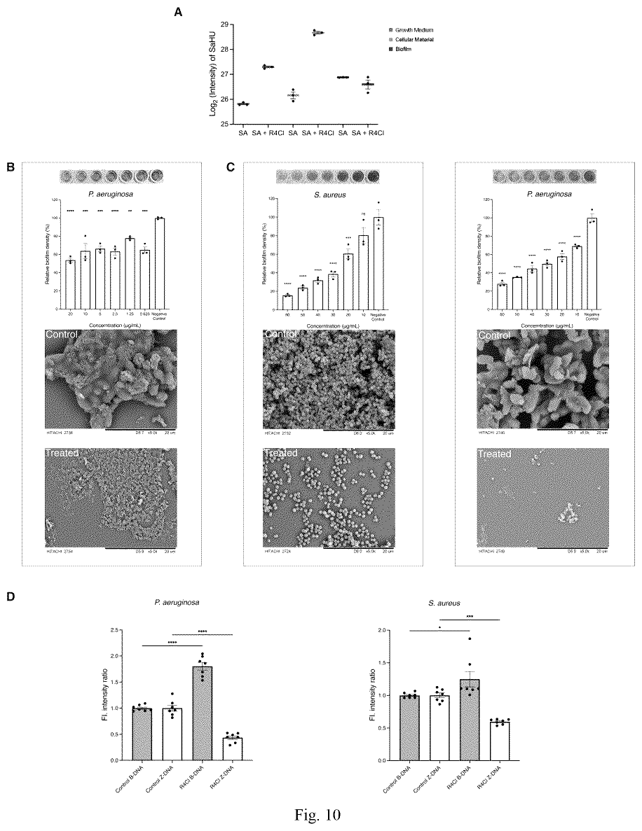

To determine whether cinnamic-hydroxamic-acid derivatives (CHADs) can be trapped extracellularly, the distribution of HU was examined under in vitro conditions. In the AST, HU exists in three distinct forms: cellular, growth medium-associated, and biofilm-associated. This differs from in vivo settings, where extracellular free HU is not physically confined as in culture plates, and the protein remains predominantly intracellular. To assess HU distribution across these three fractions, S. aureus was treated with or without R4Cl, and HU levels in each compartment were quantified using mass spectrometry (MS). Results revealed that HU was more abundant in extracellular forms—particularly in biofilm—than inside cells (FIG. 10, A), with the highest levels detected in the biofilm matrix. Treatment with a subinhibitory concentration of R4Cl (0.5×MIC, 15 μg/mL) did not alter the predominantly extracellular localization of HU, but led to a redistribution among extracellular pools: biofilm-associated HU decreased most markedly. Importantly, bacterial counts were comparable between treated and untreated cultures, confirming that the observed redistribution was not attributable to growth inhibition. These findings help reconcile the discrepancies between in vivo and in vitro observations and suggest that R4Cl may exert anti-biofilm activity, as indicated by the reduction in biofilm-associated HU alongside increases in intracellular and medium-localized HU.

Although CHAD shows negligible antibacterial activity against P. aeruginosa, it is hypothesized that they might still affect biofilm formation. To evaluate this possibility without interference from antibacterial effects, R4Cl was applied at a non-inhibitory concentration (20 μg/mL, 101 μM). When introduced at the time of bacterial inoculation, R4Cl inhibited P. aeruginosa biofilm formation at concentrations below 20 μg/mL, as shown by crystal violet staining (FIG. 10, B). Scanning electron microscopy (SEM) further confirmed the attenuation of biofilm architecture. The biofilm eradication capacity of R4Cl was also assessed by adding the compound after biofilm maturation. R4Cl exhibited potent biofilm-dispersing activity against S. aureus, causing over 60% eradication at 1×MIC (30 μg/mL) and over 80% at 2×MIC (60 μg/mL) (FIG. 10, C). A similar disruptive effect was observed against preformed P. aeruginosa biofilms, despite the lack of direct antibacterial activity against this organism. SEM results corroborated the crystal violet data, showing clear structural disintegration in biofilms formed by both species.

Within biofilms, Z-DNA architecture is stabilized by DNABII family proteins such as HU and IHF. Since CHADs disrupt HU-DNA interactions, they may also destabilize Z-DNA, thereby compromising biofilm integrity. To monitor potential B-to-Z DNA transitions in response to R4Cl, immunofluorescence staining was performed using B-DNA- and Z-DNA-specific antibodies in preformed P. aeruginosa and S. aureus biofilms, following established protocols in G. A. S. Minero, A. Mølebjerg, C. Thiesen, M. I. Johansen, N. P. Jøgensen, V. Birkedal, D. E. Otzen, R. L. Meyer, Nucleic Acids Res. 2024, 52, 1575. After normalizing to biofilm biomass (FM4-64 signal), a consistent shift was detected in both biofilm types: R4Cl treatment increased the proportion of B-DNA while significantly reducing Z-DNA content (FIG. 10, D), indicating destabilization of Z-DNA structure. These results align with the role of HU in Z-DNA stabilization and demonstrate that R4Cl interferes with HU-DNA binding.

Collectively, these data establish R4Cl—an HU inhibitor—as an effective anti-biofilm agent. It not only inhibits biofilm formation but also disrupts pre-existing biofilms, providing a new strategy for the treatment of persistent infections caused by biofilms.

### Example 9: Intervention of R4Cl in Bacterial Superhelical Structure

As an inhibitor of HU, R4Cl should theoretically inhibit the formation of HU-mediated superhelical structures. This Example verified this hypothesis.

Specifically, two bacterial adaptability-related genes, MsyB and HU in E. coli, were knocked out individually. Both of them can prevent bacteria from responding to external stress by adjusting the degree of supercoiling. The results showed that, unlike bacteria with MsyB knockout, bacteria with HU knockout exhibited an overall increase in the degree of supercoiling; whereas the introduction of HU could inhibit the formation of superhelical structures. This result indicates that HU is a gene that inhibits the formation of superhelical structures.

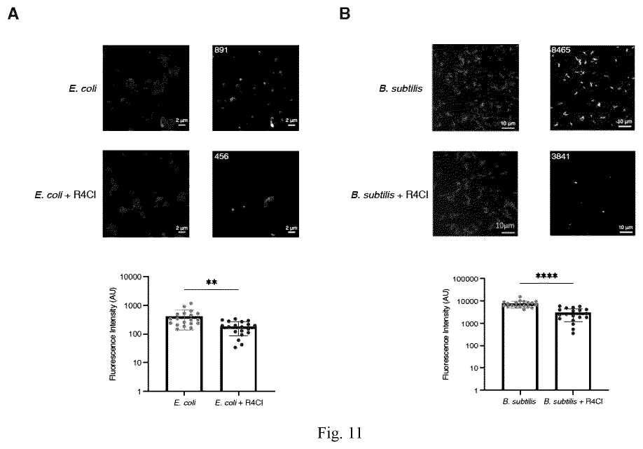

Furthermore, E. coli and Bacillus subtilis were treated with R4Cl, and an untreated group was set as the control. FIG. 11 shows the DNA superhelical structures of E. coli (A) and Bacillus subtilis (B) after R4Cl treatment. As can be seen from FIG. 11, compared with the control group, the degree of DNA supercoiling in E. coli (A) and Bacillus subtilis (B) treated with R4Cl was significantly reduced. This confirms that R4Cl functions as an inhibitor of HU and inhibits the formation of HU-mediated superhelical structures.

Example 10: Synergistic Enhancement of R4Cl in Combination with Other Antibiotics

Based on the importance of superhelical structures in bacterial adaptation to antibiotics, inhibiting the adjustment of superhelical structures should theoretically suppress the resistance response of bacteria to other antibiotics. This Example verified this using the E. coli K-12 strain MG1655.

Specifically, MG1655 was treated with R4Cl, ampicillin (purchased from Company Beecham, CB), or their combination, separately. The bacterial morphology and transcriptome expression pattern were analyzed.

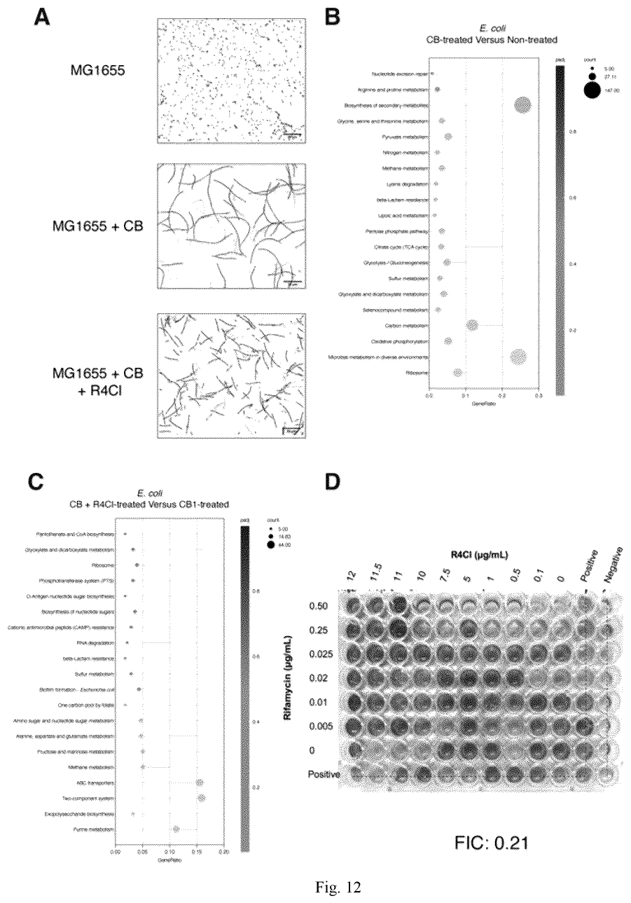

Panel A of FIG. 12 shows the morphology of MG1655 after treatment with R4Cl, ampicillin (CB), or their combination. The results indicate that R4Cl-mediated alleviation of DNA supercoiling can weaken the response of E. coli to antibiotic stress.

Panels B-C of FIG. 12 show the transcriptome analysis results of MG1655 after treatment with R4Cl, ampicillin (CB), or their combination. The results show that in the presence of R4Cl, the gene expression pattern of MG1655 changes, which can reduce the expression level of bacterial resistance genes to other antibiotics. This indicates that R4Cl can be used in combination with other antibiotics, and with the combination, it inhibits the expression of bacterial resistance genes, effectively reducing the minimum inhibitory concentration (MIC) of the combined antibiotics.

The performance of R4Cl in combination with rifampicin also confirms this point. As shown in Panel D of FIG. 12, when combined with rifampicin, R4Cl significantly reduced the MIC of rifampicin against Mycobacterium tuberculosis, demonstrating a beneficial synergistic effect in inhibiting Mycobacterium tuberculosis.

Example 11: Combination of R4Cl with Bacteriophages Prevents Bacterial Development of Bacteriophage Tolerance

Bacterial tolerance to bacteriophages is one of the hot topics in recent research, especially in the application of phage therapy—the emergence of bacterial tolerance may lead to reduced therapeutic efficacy. Due to the important role of superhelical structures in bacterial adaptive mutations, inhibiting their regulatory mechanism may help suppress the occurrence of resistant mutations. This Example combines R4Cl with bacteriophages to study its improvement effect on bacterial tolerance to bacteriophages.

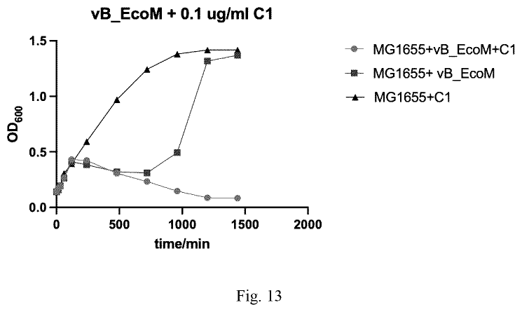

Specifically, an E. coli bacteriophage vB_EcoM with a broad lytic spectrum was combined with R4Cl (C1) to combat MG1665. The results showed that within 72 hours, in the presence of R4Cl, MG1665 failed to develop resistance to the bacteriophage (FIG. 13). This indicates that in the presence of R4Cl, the tolerance of E. coli to the bacteriophage is very low; at the same time, it shows that R4Cl can effectively prevent the formation of bacterial tolerance to bacteriophages, thereby enhancing the long-term efficacy of phage therapy. These results suggest that R4Cl can be used in combination with bacteriophages to delay the development of bacterial resistance to bacteriophages, thereby enhancing the long-term antibacterial effect of bacteriophages.

Reference throughout this specification to “an embodiment,” “some embodiments,” “one example”, “a specific example,” or “some examples,” means that a particular feature, structure, material, or characteristic described in connection with the embodiment or example is included in at least one embodiment or example of the present disclosure.

Although explanatory embodiments have been shown and described, it would be appreciated by those skilled in the art that the above embodiments are merely explanatory, and cannot be construed to limit the present disclosure, and changes, alternatives, and modifications can be made in the embodiments in the scope of the present disclosure.

What is claimed is:

1. A method for binding to a Pit domain of a bacterial histone HU to treat a bacterial infection or a disease caused by the bacterial infection, comprising administering to a subject in need a therapeutically effective amount of a cinnamobactin with the following structural formula or a pharmaceutically acceptable salt thereof: 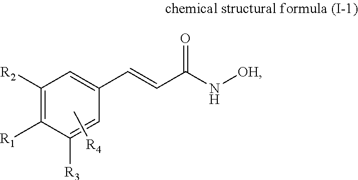
wherein
R₁ is hydrogen, fluorine, chlorine, bromine, nitryl, methoxy, aldehyde, carboxyl, 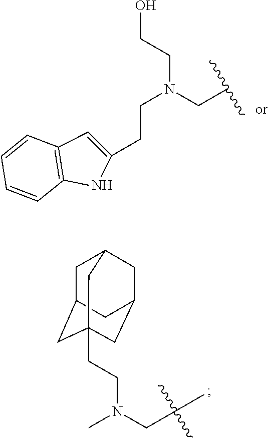 [SMILES: *CN(CCO)CCc1cc2ccccc2[nH]1 ; *CN(C)CCC12CC3CC(CC(C3)C1)C2]
R₂ and R₃ are hydrogen; and
R₄ is selected from hydrogen, fluorine, chlorine, bromine or iodine.

2. The method according to claim 1, wherein
R₁ is hydrogen, fluorine, chlorine or bromine.

3. The method according to claim 1, wherein the cinnamobactin is at least one of the following compounds:

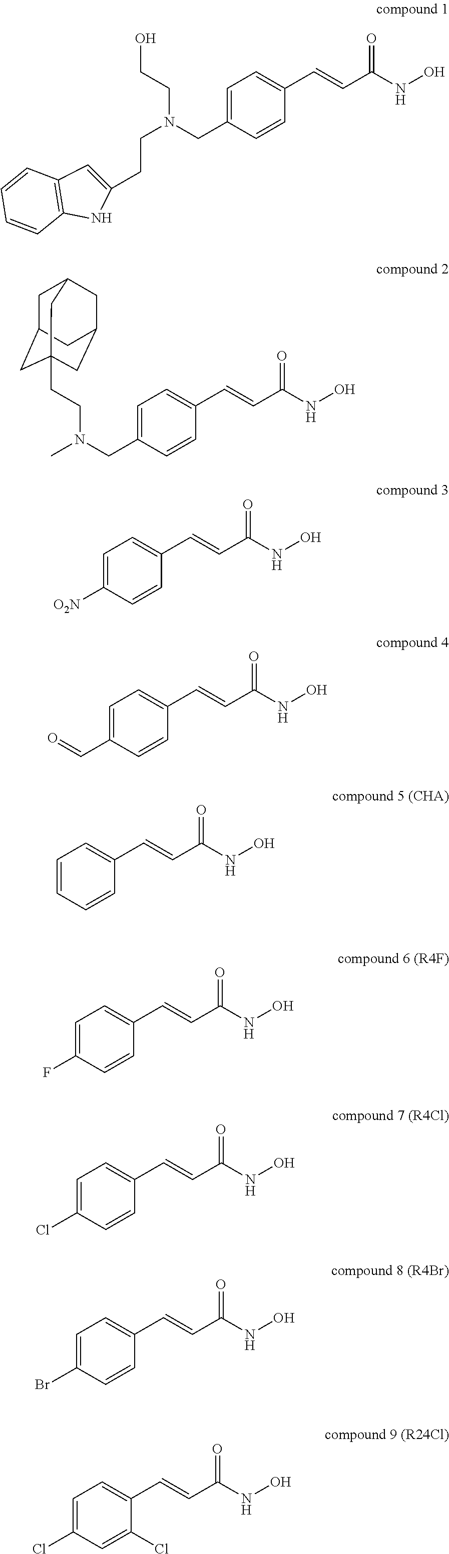

4. The method according to claim 1, wherein the cinnamobactin is compound 7 (R4Cl) or compound 9 (R24Cl).

5. The method according to claim 1, wherein the bacteria are at least one of Staphylococcus aureus, methicillin-resistant and multidrug-resistant Staphylococcus aureus, Mycobacterium tuberculosis, multidrug-resistant Mycobacterium tuberculosis, Bacillus subtilis, Escherichia coli, Klebsiella pneumoniae, Acinetobacter baumannii, and Pseudomonas aeruginosa.

6. The method according to claim 1, wherein the cinnamobactin is with a minimal inhibitory concentration of less than 250 μg/ml against the bacteria.

7. The method according to claim 1, comprising administering to the subject in need a therapeutically effective amount of the cinnamobactin or a pharmaceutically acceptable salt thereof by oral administration, subcutaneous injection, peritoneal administration, intravenous injection, intramuscular injection, intradural injection, sublingual administration, buccal administration, intrarectal insertion, intravaginal injection, ocular administration, ear administration, nasal administration, inhalation, spraying via the mouth or nose, skin administration or transdermal administration.

8. The method according to claim 1, wherein the cinnamobactin or a pharmaceutically acceptable salt thereof is administered 1 to 5 times a day.

9. The method according to claim 1, wherein the subject is a mammal.

10. The method according to claim 1, further comprising: administering an additional therapeutic agent to the subject, simultaneously or sequentially with the cinnamobactin or a pharmaceutically acceptable salt thereof, wherein the additional therapeutic agent is effective against the bacteria.

11. The method according to claim 10, wherein the additional therapeutic agent comprises an antibiotic or a bacteriophage.

12. The method according to claim 11, wherein the antibiotic comprises ampicillin or rifampicin.

13. A compound with the following chemical structural formula or a pharmaceutically acceptable salt thereof, wherein the compound binds to a Pit domain of a bacterial histone HU to treat a bacterial infection or a disease caused by the bacterial infection: 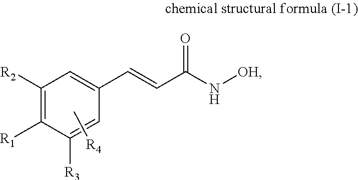
wherein
R₁ is hydrogen, fluorine, chlorine, bromine, nitryl, methoxy, aldehyde, carboxyl, 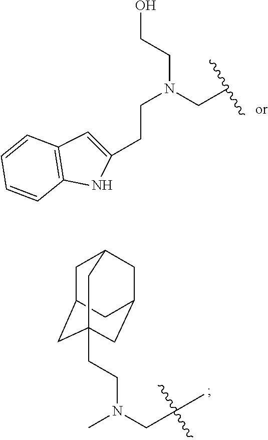 [SMILES: *CN(CCO)CCc1cc2ccccc2[nH]1 ; *CN(C)CCC12CC3CC(CC(C3)C1)C2]
R₂ and R₃ are hydrogen; and
R₄ is selected from hydrogen, fluorine, chlorine, bromine or iodine.

14. The compound according to claim 13, wherein
R₁ is hydrogen, fluorine, chlorine or bromine.

15. The compound according to claim 13, wherein the cinnamobactin is at least one of the following compounds:

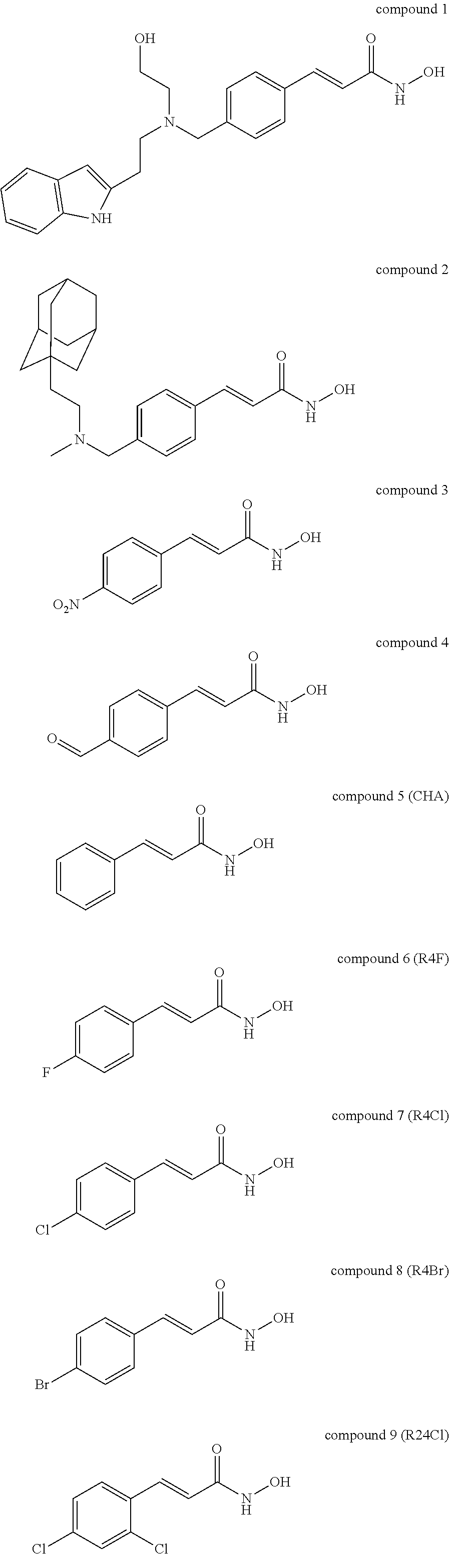

16. The compound according to claim 13, wherein the cinnamobactin is compound 7 (R4Cl) or compound 9 (R24Cl).

17. The compound according to claim 13, wherein the bacteria are at least one of Staphylococcus aureus, methicillin-resistant and multidrug-resistant Staphylococcus aureus, Mycobacterium tuberculosis, multidrug-resistant Mycobacterium tuberculosis, Bacillus subtilis, Escherichia coli, Klebsiella pneumoniae, Acinetobacter baumannii, and Pseudomonas aeruginosa.

18. The compound according to claim 13, wherein the cinnamobactin is with a minimal inhibitory concentration of less than 250 μg/ml against the bacteria.

19. A pharmaceutical composition, comprising a compound or a pharmaceutically acceptable salt thereof according to claim 13 and a pharmaceutically acceptable carrier.
